(19) United States
(12) Patent Application Publication
(10) Pub. No.: US 2020/0300230 A1
(43) Pub. Date: Sep. 24, 2020

(54) A DEVICE FOR CONVERTING THERMAL ENERGY, A CORRESPONDING SOLAR REACTOR AND RELATED PLANT

(71) Applicant: Archimede Sistemi Industriali S.r.l.s., Caltanissetta (IT)

(72) Inventors: Gianluca TUMMINELLI, Caltanissetta (IT); Gaetano TUZZOLINO, Caltanissetta (IT); Calogero GATTUSO, Caltanissetta (IT); Alessandro LUCE, Caltanissetta (IT); Roberto RIZZO, Caltanissetta (IT); Fabio SANTORO, Caltanissetta (IT)

(21) Appl. No.: 16/650,110

(22) PCT Filed: Sep. 27, 2018
(86) PCT No.: PCT/IB2018/057495

§ 371 Date: Mar. 24, 2020

(30) Foreign Application Priority Data
Sep. 28, 2017 (IT) 102017000109097

(51) Int. Cl. F03G 6/00; F24S 10/40; F24S 80/10; F24S 20/20; F24S 10/70; F23G 5/02; F24S 70/65; C07C 7/10

(52) U.S. Cl. CPC F03G 6/003; F24S 10/45; F24S 80/10; C07C 7/10; F24S 10/742; F23G 5/02; F24S 70/65; F24S 20/20

(57) ABSTRACT

Described is a solar-energy converter device including a shell and a core inside the shell, wherein the shell and the core develop axially along a longitudinal axis and include a volume therebetween. The core includes a thermally conductive matrix in a thermal exchange relationship with the volume, the matrix housing one or more flow conduits for a working fluid, the one or more flow conduits being in thermal exchange relationship with the matrix. Moreover, described is a corresponding solar reactor and a corresponding plant.

### FIELD OF THE INVENTION

The present invention relates to energy-conversion plants and to the corresponding components, in particular to plants for exploiting and converting solar energy.

### PRIOR ART

Current technology based upon plants for producing electrical energy from the thermodynamic solar source is struggling to take root, and consequently the unit production costs are far from being those typical of a mature technology. One of perhaps the most significant data available regards the fact that in roughly twenty years the production costs for these plants has dropped by barely ten times.

Thermodynamic solar plants indirectly convert solar energy into electrical energy through two distinct energy-conversion steps.

The first step consists in the conversion of solar energy into thermal energy stored in a thermovector fluid.

The second step typically consists in conversion of the thermal energy into electrical energy by means of a conventional thermodynamic cycle, for example an organic Rankine cycle (ORC).

In the plants with linear parabolic collectors referred to herein, the radiation is concentrated by means of mirrors having a linear parabolic shape on a receiver tube set on the focal line of the collector and in which a thermovector fluid, for example diathermic oil, which is heated at high temperature, flows. The thermovector fluid feeds a steam generator, which then, in turn, supplies a steam turbine or alternatively a turbogenerator of an ORC plant, converting, with high efficiency, the thermal energy supplied by the thermovector fluid into electrical energy.

To overcome the variability of the flow of energy supplied by the solar source, in addition to the possibility of storing thermal energy in thermal storage devices, it is possible to resort to thermal integration with a boiler fired by methane gas or other fuels, thus creating a hybrid plant.

Consequently, the methane-fired boiler provides an energy contribution by working simultaneously with the solar plant in daytime hours and in the case of low direct radiation, in addition to being used for restarting from a plan-downtime condition.

In other words, the conventional conversion process is summarized in the following steps:

the solar collectors continuously follow the sun, via tracking algorithms, constantly concentrating solar radiation on the receiver tube installed in the focal point of the paraboloid;

the thermal energy thus accumulated is transferred to the thermovector fluid, and from this to the working fluid of the turbogenerator, with consequent production of electrical energy, which is sent into the national electrical grid at 20 kV.

In parallel to the solar plant, the boiler integrates the amount of thermal energy necessary to bring the thermovector fluid up to working temperature, and moreover any possible process heat produced by third parties, coming from sources of a fossil and/or renewable nature, contributes to heating the thermovector fluid.

A traditional thermodynamic solar plant is hence basically constituted by:
a solar-capture system
a receiver tube
a network of conduits
a thermal-storage system
an ORC turbogenerator
a cooling system
an integration boiler
valves, pumps, and accessories

The current cost of such plant is in the region of € 8,000,000 per electrical megawatt. This is a value that is not competitive with the typical ones, for example of photovoltaic systems, this without considering the clearly higher complexity and maintenance costs, which are also higher.

There is hence the need to render the technology attractive from the technical and economic standpoint as compared to systems that are already mature, such as ones of a wind-power type, a photovoltaic type, a biomass type, etc.

A technical-economic optimisation would open the door to a wide diffusion of this technology in so far as, inter alia, the best thermodynamic solar plants are better suited to industrial uses as compared to other technologies that exploit renewable energy sources, even when simultaneously with production of electrical energy from the solar source, also high-quality heat, at high temperature, is produced.

The industrial uses and requirements are in fact oriented to a programmed production of energy, with possible differed use afforded with the aid of systems for accumulating thermal energy that are already mature and in general less expensive than accumulators of electrical energy (batteries).

If, however, it were possible to exploit better the thermal energy even before its conversion into electrical energy for industrial use, thermodynamic (TD) systems could express new and interesting potential, above all in a perspective of environmental safeguarding.

### OBJECT OF THE INVENTION

The objects of the present invention are multiple. A first object of the invention is to provide a solar-energy converter device and an associated solar reactor that will enable a broader-spectrum treatment of a fluid that is flowing through it (bearing in mind that the quality of the heat is higher, the higher the temperature of the thermovector fluid). In particular, the aim is to provide an energy-converter device capable both of carrying out thermal conditioning (albeit extended to fluids that have a high temperature and pressure and/or are chemically aggressive) and of promoting chemical reactions for treatment of the fluid itself.

A further object of the invention is to convert the thermal energy into a form of energy that can be easily stored already upstream of its subsequent conversion into electrical energy.

### SUMMARY OF THE INVENTION

The objects of the invention are achieved by a solar-energy converter device, a solar reactor, and a plant according to the annexed claims, which form an integral part of the technical disclosure provided herein in relation to the invention.

### BRIEF DESCRIPTION OF THE DRAWINGS

The invention will now be described with reference to the annexed drawings, which are provided purely by way of non-limiting example, and in which:

FIG. 1 is an overall view of an energy-converter device according to various embodiments of the invention, and in particular herein represented according to a first embodiment of the invention itself;

FIG. 2A is a detailed view of the energy-converter device according to the first embodiment and according to the arrow A of FIG. 1, whereas FIG. 2B is an exploded view of a component of the energy-converter device of FIG. 2A;

FIG. 3A is a detailed view of an energy-converter device according to a second embodiment (once again referring the area associated to the arrow A of FIG. 1), whereas FIG. 3B is an exploded view of a component of the energy-converter device of FIG. 3A;

FIG. 4A is a detailed view of an energy-converter device according to a third embodiment (once again referring to the area associated to the arrow A of FIG. 1), FIG. 4B is a view of a component, in the assembled condition, of the energy-converter device of FIG. 4A, whereas FIGS. 4C and 4D illustrate elements constituting the component of FIG. 4A;

FIG. 5A is a detailed view of an energy-converter device according to a fourth embodiment (once again referring to the area associated to the arrow A of FIG. 1), FIG. 5B is a view of a component, in the assembled condition, of the energy-converter device of FIG. 5A, and FIG. 5C is an exploded view of a portion of the components of FIG. 5B;

FIG. 6A is a detailed view of an energy-converter device according to a fifth embodiment (once again referring to the area associated to the arrow A of FIG. 1), whereas FIG. 6B is a detailed view referring to the circle B of FIG. 6A;

FIG. 7 is a schematic view of a solar reactor according to a preferred embodiment of the invention; and

FIG. 8 illustrates a plant that uses the energy-converter device and the solar reactor according to the invention.

### DETAILED DESCRIPTION OF EMBODIMENTS OF THE INVENTION

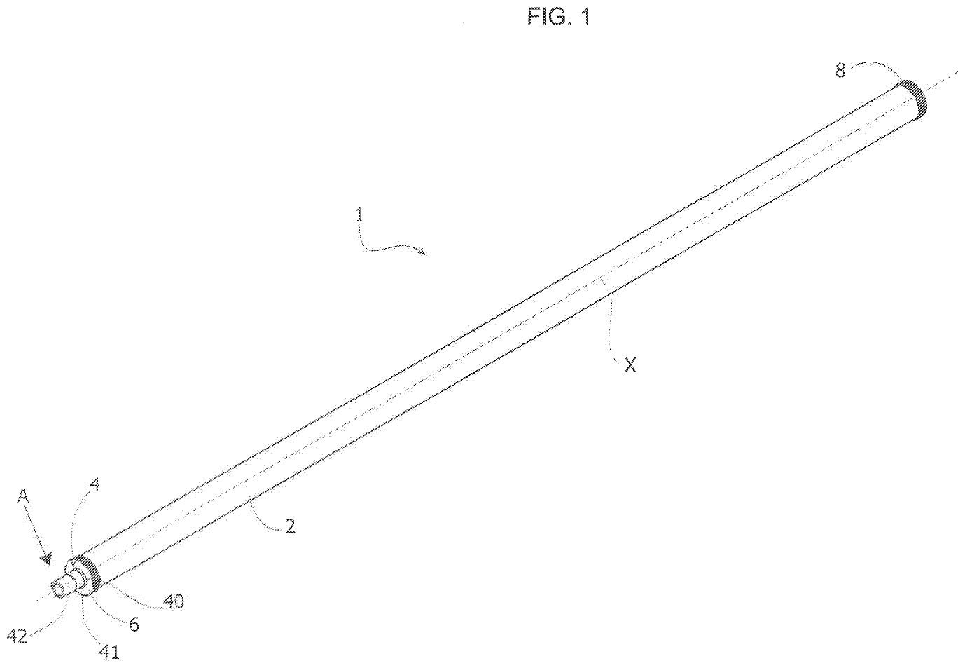

Reference number 1 in FIG. 1 designates as a whole a solar-energy converter device according to various embodiments of the invention, and here in particular according to a first embodiment.

The converter device 1 includes a shell 2 and a core 4, both developing axially along a longitudinal axis X and being coaxial to one another and to the axis X. Set at the ends of the converter 1 are a first end plate 6 and a second end plate 8, which delimit the converter and enclose in a fluid-tight way a volume comprised between the shell 2 and the core 4. Preferentially, the converter device 1 has an overall cylindrical shape so that both the shell 2 and the core 4 have an overall cylindrical shape. In this case, the volume comprised between the core 4 and the shell 2 is shaped like a cylindrical annulus. In the volume in question, vacuum is obtained.

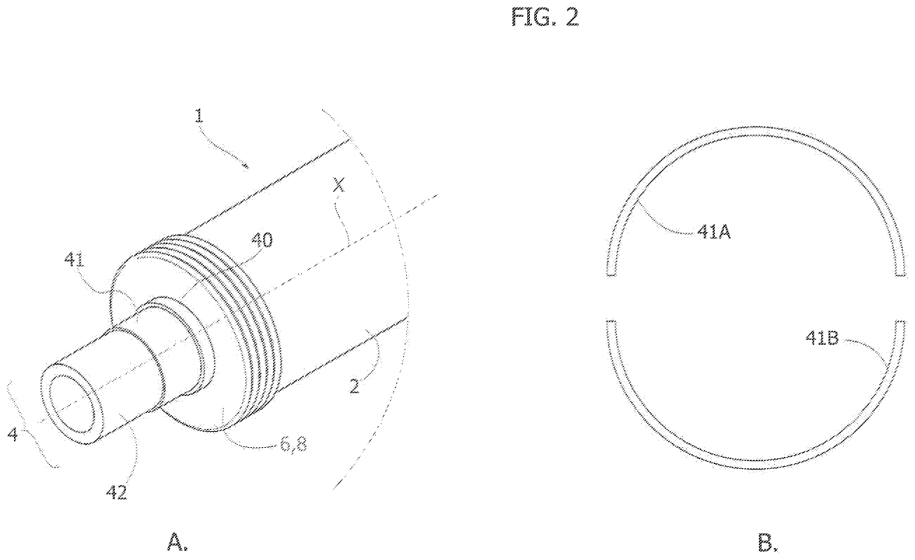

In the preferred embodiments, the core 4 includes a tubular element 40, housed within which is a matrix 41, which in turn houses one or more flow conduits for a working fluid that are in a relation of thermal exchange with the matrix itself. Present in the embodiment of FIG. 1 and FIG. 2A is a single flow conduit 42. All the elements in point have a cylindrical shape and are coaxial to the axis X. All the elements in point are moreover fitted inside one another (conduit 42 within the matrix 41, and matrix 41 within the tubular element 40) with minimum play, only necessary to enable assembly. The matrix 41 is moreover in a relation of thermal exchange with the volume comprised between the core 4 and the shell 2, within which vacuum is obtained (meaning thereby a condition as close as possible to the theoretical vacuum).

It will be appreciated, on the other hand, that in the preferred embodiments the converter device 1 can be obtained starting from a common receiver tube for thermodynamic solar plants. The base structure of the aforesaid device includes, in fact, the shell 2 (outer tubular element) and the inner tubular element 40, as well as the end plates 6 and 8. For this reason, exactly as in traditional receiver tubes for thermodynamic solar plants, the shell 2 of the converter 1 is provided on the outside with a high-absorbency coating (for example, a selective high-absorbency dark paint) for maximisation of the radiant energy absorbed, and is made of glass. The tubular element 40 is, instead, made of (thermally conductive) metal material such as steel, and the vacuum is obtained between the tubular element 40 and the shell 2. The differences in thermal expansion between the shell 2 (glass) and the tubular element 40 are compensated by the end plates 6 and 8.

Basically, the converter 1 according to the invention is preferentially obtained by insertion of the matrix 41 and of the respective one or more flow conduits 42 within the tubular element 40 of a common receiver tube for thermodynamic solar plants. This enables reduction of the costs of production of the converter. Notwithstanding this, especially for some applications, the converter 1 can be obtained regardless of pre-existing componentry. In this case, according to the design requirements, the tubular element 40 may be omitted, and the matrix 41 may directly face the shell 2. In any case, the matrix 41 is in a relation of thermal exchange with the volume comprised between the core and the shell, either directly, in the case where the tubular element 40 is omitted, or by way of the tubular element 40, in the case where this is provided (preferred embodiments).

Hence, more in general the following configuration (which is applicable also to the other embodiments that will be described) may apply:
the shell 2 is a tubular shell;
the matrix 41 extends along the longitudinal axis X from a first end to a second end of the tubular shell 2, in the case where this is envisaged; this also applies to the tubular element 40;
the one or more flow conduits extend within the matrix along the longitudinal axis X and come out from said first and second ends; and
the first end plate 6 and the second end plate 8 are arranged, respectively, at said first and second ends and delimit the volume between the tubular shell 2 and the core 4; within this volume, vacuum is obtained (i.e., it is a volume emptied of the gas/air present therein).

With reference to FIG. 2B, the matrix 41 is made of solid material with high thermal conductivity (not lower than 50 W/(m·K), more preferably not lower than 140 W/(m·K), and even more preferably not lower than 220 W/(m·K)), such as copper, graphite, or aluminium, and is provided in a number of sections to facilitate assembly. In particular, in the embodiment of FIGS. 1 and 2, the matrix 41 is provided in two C-shaped half-sections 41A, 41B that are set up against one another to form a matrix shaped like a cylindrical annulus that receives the single flow conduit 42.

The flow conduit 42 is made of a material that is resistant to high pressures, high temperatures, and chemical aggression, such as Inconel, or other metal alloys of similar performance. This renders the converter 1 suited to being used with fluids at high pressure and/or temperature, and even with aggressive chemical species. This means that it becomes possible to treat not only low-pressure fluids, as in the case of traditional receiver tubes of thermodynamic solar plants, but also high-pressure fluids, such as pressurised water or steam, and to allow the development of chemical reactions, such as gasification in supercritical water. Even though the converter 1 may be used in a way identical to a receiver tube, it is in effect a multipurpose reactor.

In this embodiment, as may be appreciated from FIG. 2A, the matrix 41 has as a whole a thin wall, whereas the flow conduit 42 has a thick wall.

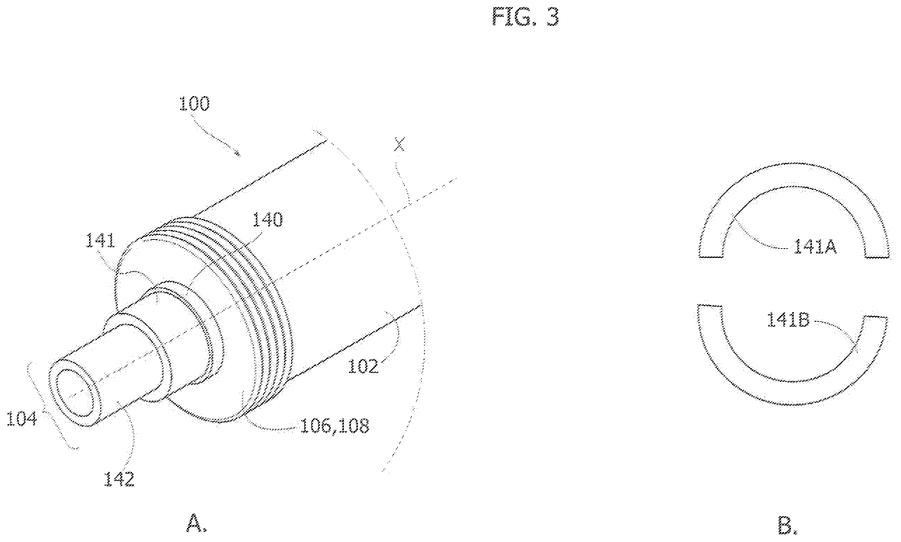

With reference to FIGS. 3A and 3B, a second embodiment of the converter according to the invention is designated by the reference number 100. All the components that are functionally (and, where applicable, structurally) identical to the ones already described as regards the converter 1, are designated by the same reference numbers increased by 100. Hence, designated by 102 is the shell, by 104 is the core, by 106 and 108 are the first and second end plates, by 140 is the inner tubular element (where envisaged), by 141 is the matrix, by 141A and 141B are the half-sections, and by 142 is the flow conduit.

For this reason, the same structural and functional description already provided previously applies except where otherwise specified. In this regard, the embodiment in question differs from the converter 1 only in that now it is the matrix 141 that has a thick wall, whereas the flow conduit 142 has a thin wall.

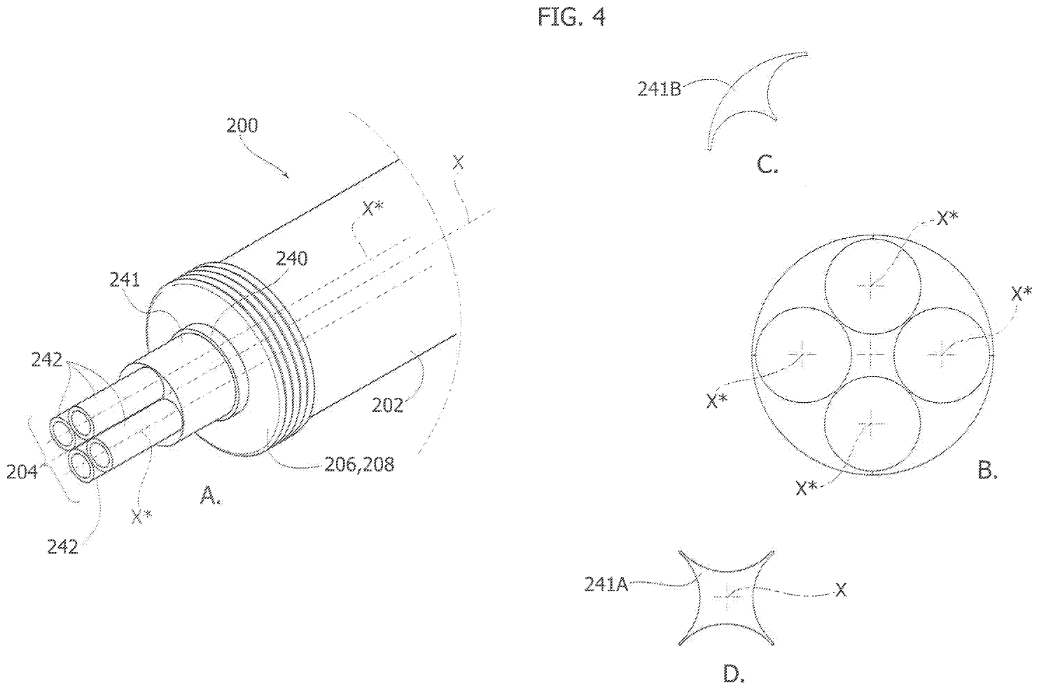

With reference to FIGS. 4A and 4B, a third embodiment of the converter according to the invention is designated by the reference number 200. All the components that are functionally (and, where applicable, structurally) identical to the ones already described as regards the converters 1, 100 are designated by the same reference numbers increased by 200. Hence, designated by 202 is the shell, by 204 is the core, by 206 and 208 are the first and second end plates, by 240 is the inner tubular element (where envisaged), by 241 is the matrix, and by 242 is the flow conduit (in this case, more than one).

For this reason, the same structural and functional description already provided previously applies, except where otherwise specified. In this regard, the embodiment in question differs from the converters 1, 100 only in that it includes a plurality of flow conduits 242, here in particular four arranged at the vertices of a square. The matrix 241 consequently includes four axial channels, each with axis X* parallel to the axis X, housed within which are corresponding flow conduits 242 (which are also coaxial to the respective axes X*).

Conveniently, the matrix 241 is obtained by setting modular elements side by side. In the preferred embodiment (see FIGS. 4B, 4C, and 4D), the matrix 241 is assembled using two types of modular elements, in particular a profile with star-shaped cross section 241A (FIG. 4D) and a profile with double-sickle cross section 241B (FIG. 4C). By assembling four profiles 241B in a circle, and setting one profile 241A coaxially to the axis X (located at the centre of the circle) the section with four holes of the matrix is obtained (visible in FIG. 4B).

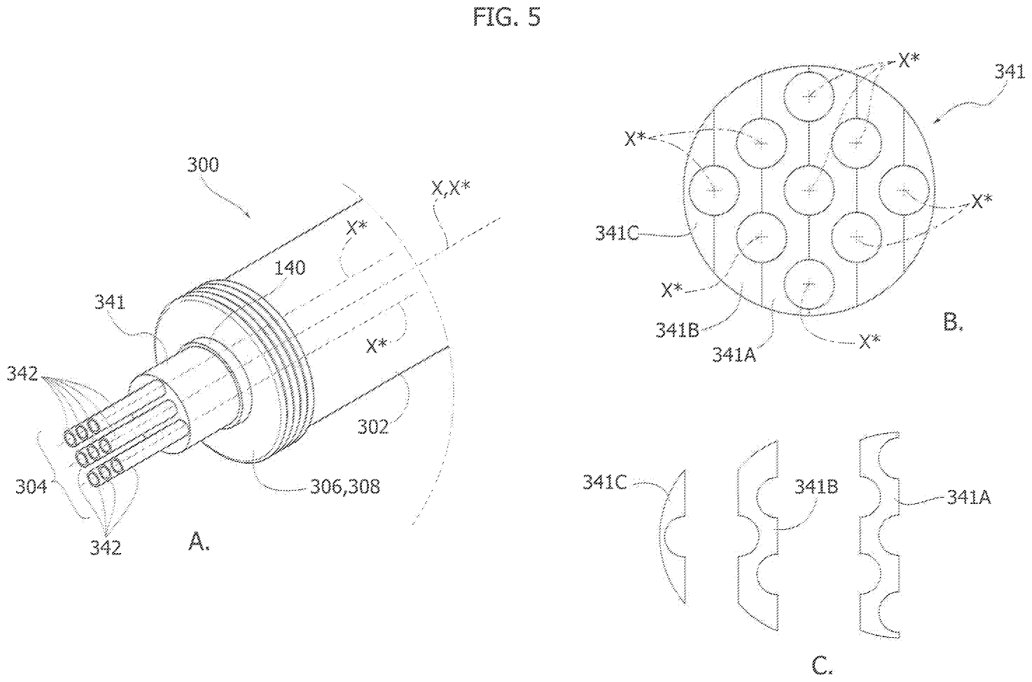

With reference to FIGS. 5A and 5B, a fourth embodiment of the converter according to the invention is designated by the reference number 300. All the components that are functionally (and, where applicable, structurally) identical to the ones already described as regards the converters 1, 100, 200 are designated by the same reference numbers increased by 300. Hence designated by 302 is the shell, by 304 is the core, by 306 and 308 are the first and second end plates, by 340 is the inner tubular element (where envisaged), by 341 is the matrix, and by 342 is the flow conduit (in this case, more than one).

For this reason, the same structural and functional description already provided previously applies, except where otherwise specified. In this regard, the embodiment in question differs from the converters 1, 100 only in that it includes a plurality of flow conduits 342, and moreover differs from the converter 200 in the number of the flow conduits (nine instead of four, once again arranged according to a square array).

The matrix 341 consequently includes nine axial channels, each with axis X* parallel to the axis X, housed within which are corresponding flow conduits 342 (which are also coaxial to the respective axes X*).

Conveniently, the matrix 341 is obtained by setting modular elements side by side. In the preferred embodiment (see FIGS. 5B, 5C), the matrix 341 is assembled using three types of modular elements, in particular a first profile and a second profile that have a cross section shaped like a circular segment having two bases 341A, 341B, and a profile that has a cross section shaped like a circular segment having one base 341C. Each of the profiles includes, on at least one side thereof, cylindrical grooves (in numbers that differ by one) configured for being set alongside homologous grooves on another profile to define a flow channel.

By assembling the three profiles in the sequence illustrated in FIG. 5C, half of the cross section of the matrix 341 is covered so that another three profiles are necessary, arranged in a specular way, hence six profiles in all.

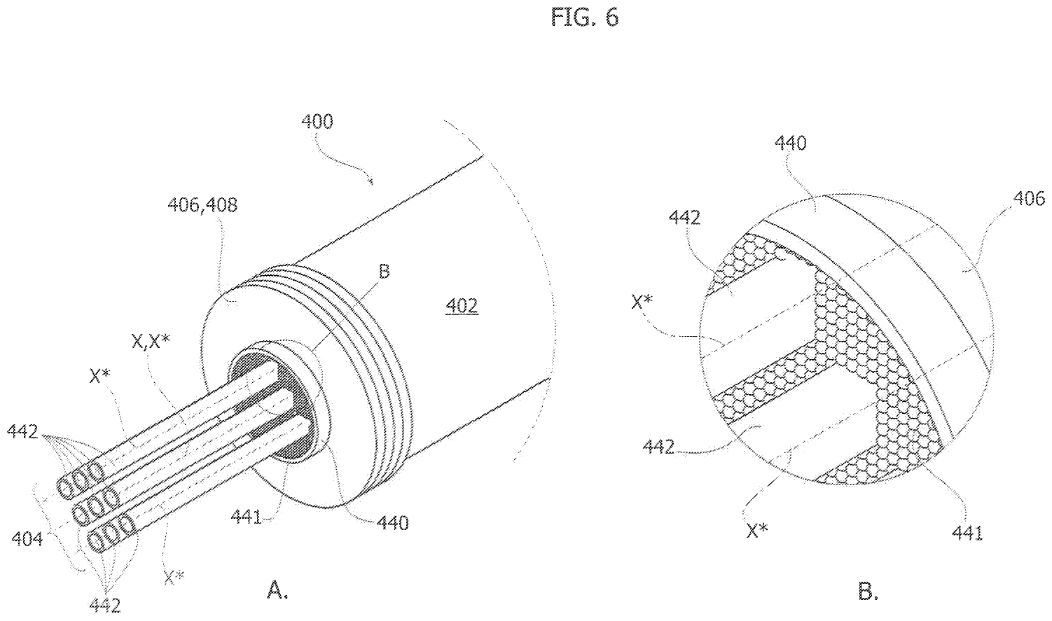

Finally, with reference to FIGS. 6A and 6B, a fifth embodiment of the converter according to the invention is designated by the reference number 400. All the components that are functionally (and, where applicable, structurally) identical to the ones already described as regards the converter 1 are designated by the same reference numbers increased by 400. Hence, designated by 402 is the shell, by 404 is the core, by 406 and 408 are the first and second end plates, by 440 is the inner tubular element (which is here always envisaged, as will be seen shortly), by 441 is the matrix, and by 442 is the flow conduit (here, more than one).

For this reason, the same structural and functional description already provided previously applies, except where otherwise specified. In this regard, the embodiment in question differs from all the converters 1, 100, 200, 300 described previously in that the matrix 441 is made of a material with high apparent thermal conductivity (not lower than 45 W/(m·K), more preferably not lower than 90 W/(m·K), and even more preferably not lower than 140 W/(m·K)), such as copper, graphite, or aluminium, with granular structure (for example, in powder form), provided that it can be easily compacted so as to minimise the degree of empty space in the volume in which the flow conduits are housed and thus ensure a good thermal conduction.

The whole picture is represented in FIG. 6B. The flow conduits 442 are hence embedded directly in the granular material in point and are arranged according to a square array, as in the case of the converter 300.

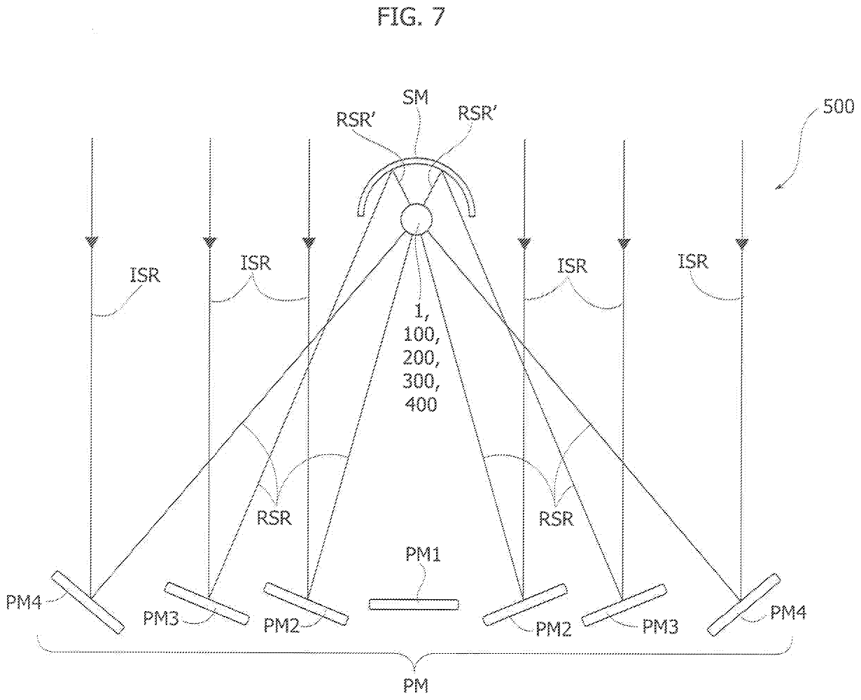

With reference to FIG. 7, the reference number 500 designates as a whole a solar reactor that uses one or more converter elements 1, 100, 200, 300, 400 according to the invention.

The solar reactor 500 is basically a solar collector, which comprises the converter 1, 100, 200, 300, 400 and a device for concentrating solar radiation including one or more reflecting elements configured for concentrating the reflected solar radiation on the solar-energy converter device 1, 100, 200, 300, 400.

In the preferred embodiment illustrated here, the device for concentrating solar radiation is of the type constituted by an array of Fresnel mirrors, and includes an array PM of primary mirrors, which are arranged symmetrically, and with a progressively increasing inclination, with respect to a central mirror PM1. Here in particular—purely by way of example—the array PM of primary mirrors includes three pairs of peripheral mirrors PM2, PM3, PM4 (listed in increasing order of distance from the mirror PM1). On a focal axis of the array PM the one or more converter elements 1, 100, 200, 300, 400 according to the invention are located, in particular by getting the axis X to coincide with the focal axis of the array PM. A secondary mirror SM is moreover set, with respect to the one or more converter elements, on a side opposite to the array PM. It is moreover to be borne in mind that, in alternative embodiments, it is possible to resort to solar-concentrator devices with paraboloidal geometry (either linear or disk-shaped), once again arranging the converters on the focal axis (in the case of a linear paraboloid) or else along the line joining the vertex and the focus (in the case of a disk-shaped paraboloid).

The one or more converter elements 1, 100, 200, 300, 400 are connected together in hydraulic series, and may moreover be suited for connection in parallel with one or more similar series forming part of one or more further collectors 500. The connection is obtained by means of threaded joints (in part provided on the end plates of the converter), or else flanged joints capable of absorbing the differences in thermal expansion that were to arise between one converter element and the next, or else between the one or more flow conduits of each converter element and the thermally conductive matrix in which the one or more flow conduits are housed. It should, on the other hand, be noted that the connection between successive converters obtains in effect a thermal interruption between the respective matrices 41, 141, 241, 341, 441 that limits the conductive heat exchanges in an axial direction, confining them, instead, within each converter to maximise the process efficiency. It should moreover be noted that, in order to prevent as much as possible conductive heat exchanges in the axial direction it is possible to provide the matrices 41, 141, 241, 341, 441 themselves within the individual converter elements 1, 100, 200, 300, 400 with a plurality of sections separated by thermal interruptions (diaphragms or gaps in which vacuum is obtained).

The converter constitutes a single repetitive modular unit of the reactor 500, which is connected, at the two ends, to as many converters until the desired total linear length is obtained, as in a plug&flow reactor (PFR). The desired total linear length is determined via a computation procedure in the design stage that takes into account, inter alia, the flowrate of waste to be treated, the environmental conditions and conditions of insolation of the place in which the reactor 500 is to operate, and the minimum stay time that it is necessary to ensure according to the treatment to be carried out (for example, as will be seen shortly, to maximise conversion of the waste). The reactor 500 may have an overall length even of some hundreds of metres, and consequently may be constituted by several hundreds of repetitive units (converters 1, 100, 200, 300, 400), which are interconnected, in a serial configuration.

Given the marked modular nature of the system, it is also possible to envisage—as has been said—an arrangement in parallel of a number of converters set in series in order to increase, in a discrete and easily predictable way, the flow rate of working fluid that can be treated by the plant.

In addition, according to an advantageous aspect of the invention, it is possible to equip the network of converters 1, 100, 200, 300, 400 (and consequently the reactor 500) with a system of automatic valves for controlling and directing the flow located between one converter (or series of converters) and the next. The automatic valves may be three-way or four-way valves, with the following modes of connection:

a) three-way valve—a first port is connected to the converter (or series of converters) upstream, a second port is connected to the converter (or series of converters) downstream, and a third port (or derivation port) is connected to a first end of a derivation conduit, the second end of which is in turn connected to a derivation port of a corresponding (three-way or four-way) valve inserted in a further series of converters, or else upstream of the further series, or again upstream of a single converter;

b) four-way valve—a first port is connected to the converter (or series of converters) upstream, a second port is connected to the converter (or series of converters) downstream, a third port and a fourth port (or derivation ports) are connected to respective first ends of two derivation conduits, the second ends of which are in turn connected to a derivation port of a corresponding (three-way or four-way) valve inserted in a further series of converters, or else upstream of the further series, or again upstream of a single converter.

Each series of converters 1, 100, 200, 300, 400 is associated to a corresponding series of solar-concentrator devices of the types specified above.

In the case of a network including a hydraulic connection in parallel of three or more series of converters, the three-way valve can be used both in the intermediate series and in the end series of the parallel (assuming series of converters that develop physically parallel to one another, the end series are the ones at the sides, i.e., the ones that are flanked by just one further series, whereas the intermediate series are the ones comprised between the sides, i.e., the ones that are flanked by two further series), whereas the four-way valve can be used chiefly in the intermediate series.

At a general level, the solar reactor 500 (which in this case is to be understood as an aggregate of simple reactors) hence includes a plurality of converter devices 1, 100, 200, 300, 400 hydraulically connected together, where the hydraulic connection includes a parallel connection of converter devices 1, 100, 200, 300, 400, which are in turn hydraulically connected in series, and where in one or more series of converter devices 1, 100, 200, 300, 400 one or more valves are inserted for controlling and directing the flow, which are configured for establishing a hydraulic connection in derivation of the corresponding series with one or more further series.

The moving element(s) of each valve is preferably controlled by means of an electrical servo motor or else pneumatically. The network of valves (and hence the valves themselves) is electronically managed in a centralised way through a computer in which, inter alia, a global monitoring of the plant in which the set of reactors 500 is used is implemented.

By controlling the aforesaid valves it is possible to obtain dynamic paths of flow according to the radiation available. This corresponds, as practical effect, to varying the length of the arrangement in series/parallel of solar receivers/converters, and hence makes it possible to have path of flows of maximum length when the radiation is minimum so as to increase the times of stay of the fluid and allow it to reach high temperatures even in hours of less radiation. When the radiation is or returns abundant, in order to guarantee a constant operating temperature (necessary to sustain the chemical reactions) it is possible to:
intervene on the system of automatic valves to obtain shorter path of flows given the same operating flow rate; or else
intervene on the flowrate of the hydraulic-supply assembly of the network of converters (for example, a supply pump) to increase the operating flowrate given the same total length of the path of flow.

As represented schematically in FIG. 7, the reactor 500 receives an incident solar radiation ISR, and reflects it towards the focal axis (reflected solar radiation RSR) via the mirrors PM1-PM4 (which are able to turn about their own axis of symmetry in such a way as to reflect and concentrate—instant by instant—always the maximum incident radiation towards the converter) in such a way that the radiation impinges upon the converter 1, 100, 200, 300, 400. The amount not directly intercepted by the converter is further reflected (radiation RSR′) by the secondary mirror SM, and hence ends up impinging upon the converter.

Operation of the converter 1, 100, 200, 300, 400, as well as of the reactor 500, is described in what follows.

The converter 1, 100, 200, 300, 400 operates substantially as a multipurpose chemical reactor. As has been said, it can indifferently operate with non-aggressive fluids at high pressure and temperature (for example, pressurised water or steam), obtaining a thermal conditioning thereof for applications in traditional thermodynamic solar plants with fluids at high temperature and pressure, or else with aggressive chemical species or in general reactive chemical species, carrying out a chemical treatment thereof in addition to the thermal treatment (for example, thermal cracking or thermal reforming or again chemical processes promoted by high temperatures, such as gasification of organic compounds in supercritical water).

In this connection, the flow conduits must be made of a material that allows them to withstand, in operation, pressures higher than the critical pressure of water (221 bar), preferably 230 bar, and even more preferably 250 bar. Moreover, the material must simultaneously withstand temperatures higher than the critical temperature of water (374° C.), preferably 550° C., and even more preferably 700° C.

Finally, as regards resistance to chemically aggressive environments, the material of which the conduits 42, 142, 242, 342, 442 of the nests of tubes 4, 104, 204, 304, 404, respectively, are made must be able to withstand chemically the action of the corrosive components (such as hydrogen sulphide or hydrochloric acid) or embrittling components (such as hydrogen) that may be formed in the process of gasification in supercritical water to which the present invention is preferably to be applied.

In both cases, the energy entering the converter is transferred by the solar radiation that impinges upon the converter itself. This realization, which is strictly related to that of common receivers for thermodynamic solar plants, inherits the mechanism of absorption of energy thereof. The incident solar radiation ISR impinges upon the shell 2, 102, 202, 304, 402 and is absorbed thanks to the coating of the latter. The presence of the vacuum between the shell 2, 102, 202, 304, 402 and the core 4, 104, 204, 304, 404 avoids any loss of energy by convection. The shell moreover withholds, reflecting it, the radiation that penetrates through it, concentrating the radiation on the core. From here, thanks to the matrix 41, 141, 241, 341, 441, the thermal energy is transferred to the fluid that flows in the one or more flow conduits 42, 142, 242, 342, 442, which may have an outer diameter of from 0.5 to 2 inches (according to the diameter chosen, it will be possible to house a different number of conduits, in any case less than ten, given the typical dimensions that can be used).

The high amount of thermal energy transmitted to the working fluid within the flow conduits 42, 142, 242, 342, 442, as well as the high resistance of the conduits to high pressures and temperatures, and to chemical aggression, renders the converter 1, 100, 200, 300, 400 suited for use as gasification reactor 1, 100, 200, 300, 400 in supercritical water, and consequently renders the reactor 500 a solar gasifier in supercritical water, which is useful, for example, for treating the organic fraction of municipal solid waste. Of course, this is only one example of possible use. As said, the reactor 500 can operate also as reactor for cracking or reforming of hydrocarbons or organic fractions of waste or again for other chemical processes promoted by high temperatures and pressures.

The process of gasification in supercritical water develops, in fact, in rather severe operating conditions in so far as it is necessary to exceed the pressure and temperature of the critical point of water (221 bar, 374° C.). The water, in these conditions, referred to as “supercritical water”, loses its properties of polar compound and hence is no longer able to solubilise ionic species. Consequently, the capacity of solubilising non-polar species, such as the organic molecules present in municipal solid waste, increases considerably. Hence, the water functions as carrier for the organic species (which would have, instead, a low solubility in normal subcritical conditions), in addition to constituting the reaction environment (given that it constitutes over 90% of the total reaction volume) and participating in the reaction itself as mild oxidising agent.

The chemistry of gasification reactions is extremely complex, both on account of the enormous variety of the chemical species possibly present in the starting waste and on account of the articulated mechanisms that involve numerous intermediate compounds of reaction and reactive stages, both endothermic ones and exothermic ones.

However, a generic gasification reaction may be expressed in the following simplified form:

CaHbOcNdXf (waste-slurry)+H₂O (wastewater/discharge water)→CH₄+H₂+CO₂+CO+H₂S+H₂O (clean water)+inert substances (salts of X, N)

where X is a generic halogen (element of Group VII) or an element of Group VI (e.g., sulphur), N is nitrogen (or else another element of Group V, e.g., phosphorus), O is oxygen, H is hydrogen, and C is carbon.

In general, the gasification reactions may be considered as reactions of partial oxidation, in which the water participates as oxidising reagent, playing a role similar to that of oxygen in normal combustion in air.

According to this global reaction, among the products of gasification it is possible to find methane, hydrogen, carbon monoxide and carbon dioxide, hydrogen sulphide (if the input waste contains sulphur), water, and inert salts.

Supercritical gasification of organic matter affords some important advantages, listed below, over conventional treatments, such as gasification with steam/air.
It can be applied, with high yields, to organic matter with high water content (up to 95%); hence, no pre-treatment of drying of the charge that would reduce the thermal efficiency of the process is required.
Since the aforesaid gasification reactions develop in diluted medium, in conditions of hydrolysis of the organic molecules, there is no the formation of carbon deposits (tar and char), which constitute a considerable limitation of conventional gasification processes.
The relatively low process temperatures (<650° C.) and the low oxygen content do not enable formation of dangerous pollutants, such as NOx, which, instead, are always formed in conventional gasification and combustion processes.
The gaseous products are obtained at high pressure, thus obtaining a recovery of the energy involved in pumping the organic charge, in the form of mechanical energy of the gases themselves, at the moment of expansion thereof downstream of the reactor.

To obtain an acceptable conversion of the organic waste it is necessary to guarantee a time of stay in the region of a few minutes of the current supplied at a temperature of between 550° C. and 650° C. From the scientific literature widely available on the subject, it is known that the conversion of the waste at input (frequently expressed in terms of “carbon efficiency”, i.e., as moles of carbon present in the gas at output over the moles of carbon at input in the feed) depends upon the nature of the waste supplied (i.e., upon its final composition) and upon the operating conditions (temperature and stay time). In particular, the composition of the current supplied and the temperature of reaction affect the thermodynamics of the process, which may be globally endothermic or exothermic, or even present athermic characteristics. The yield in products that are energetically useful in so far as they have a high calorific power (hydrogen, methane) is also a function of the operating conditions of the process, and, in the light of the information that can be found in the literature and of the results of the numerous experimental studies conducted by a large number of research groups, it is possible to state that as the temperature and/or the stay time increase/increases, the yield in progressively lighter fuels increases (in particular, the yield in hydrogen increases).

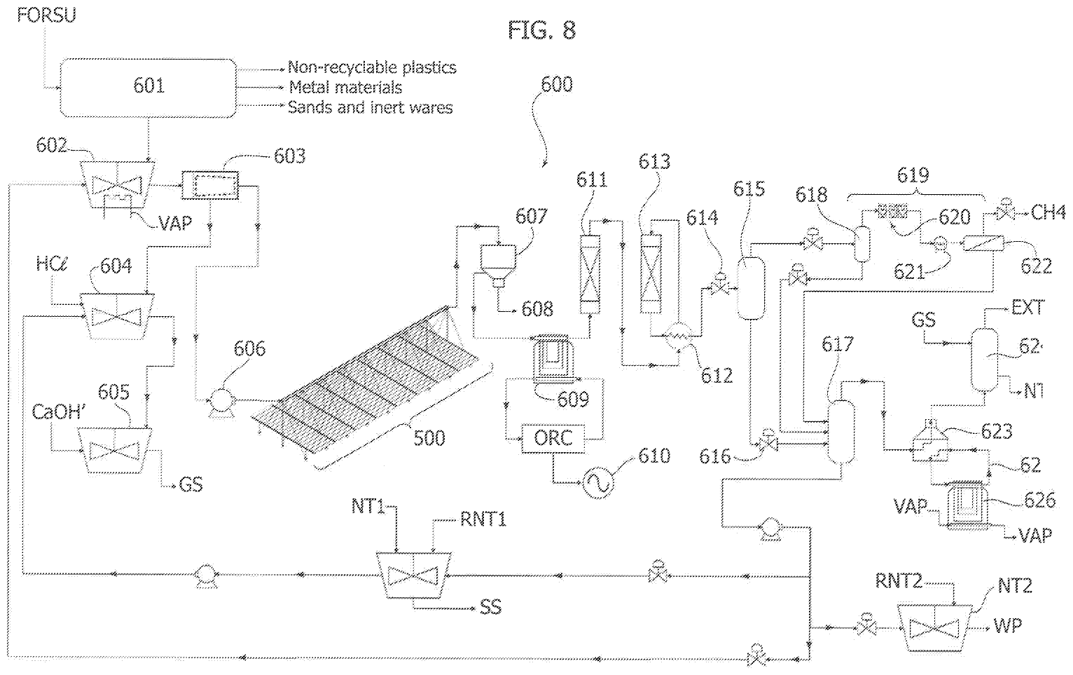

In order to convert the thermal energy into a fuel that can be easily stored and in the limit transported, the converter 1, 100, 200, 300, 400 (in particular, when it equips a solar reactor 500) is exploited in a treatment plant 600, schematically illustrated in FIG. 8. The plant 600 is specifically devised for treating the organic fraction of municipal solid waste (OPMSW).

In the plant 600, the raw food waste, which in the sector of common waste sorting is referred to as “wet organic waste” (kitchen waste, leftovers of food, waste of fruit and vegetables, small bones, egg shells, food that has gone off and expired without packaging, wooden sticks for ice-creams or lollipops, ashes and soot from fireplaces, cut flowers, coffee dregs and tea leaves and filters, non-printed paper handkerchiefs and serviettes, compostable plastic, etc.) gathered in bags is processed in a pre-treatment assembly 601 altogether similar to that of a plant for anaerobic digestion of biomass.

The bags are mechanically opened, and the contents undergo in order:

1) dynamic screening for removing approximately 9-10% of non-recyclable plastic material;

2) treatment of magnetic removal for removing possible traces of ferrous materials;

3) mechanical shredding, downstream of which approximately a further 5% of plastic material is removed; and

4) degritting for removing inert sands and solids, heavier than the organic compounds, which are a potential cause of wear of the mechanical parts.

The product at output from the mechanical pre-treatment has the consistency of sludge and is sent, by dispensing a make-up of water recycled by an atmospheric separator (see below), to a thermal-hydrolysis tank 602, supplied with low-pressure steam VAP, the purpose of which is to degrade the more complex organic molecules (e.g., lignin or cells of the sludge).

The capacity of the tank is designed so as to ensure short times for loading the daily ration of OFMSW, without sensibly altering the quality of the mixture, thus enabling subsequent supply of a constant flow of homogeneous product to a centrifugal separator 603 located immediately downstream.

In the separator 603 the solid organic components that have not been solubilised and hydrolysed in the thermal-hydrolysis tank 602 are separated and are then fed to a second hydrolysis tank 604, which operates in an acid environment (HCl solution), in which hydrolytic demolition of proteins, carbohydrates, and lipids occurs, with final production of sugars, amino acids, and volatile acids with a low number of carbon atoms.

The solution then passes into a third hydrolysis tank 605, operating this time in a basic environment, dispensed in which is an amount of lime (CaOH) that favours passage into solution of the organic solids, not dissolved in the acid environment.

The solution, which is by now basic, is drawn off and sent as scrubbing agent GS to the column for scrubbing the gases discharged into the atmosphere (see below), before ending up in a fourth tank NT1, or neutralisation tank, in which neutralisation reagents (flow RNT1) are added. Here, the inert salts precipitate and are removed (flow SS), while the aqueous solution is sent back as recirculation current to the acid-hydrolysis tank 604.

From the centrifugal separator 603 the solution to be gasified is sent to the solar reactor for gasification in supercritical water 500 via a pump 606, in particular a volumetric pump for slurry, which continuously draws off the nominal process flow rate. This flow rate may even be variable (within the operating range for which the system has been designed), or else may be maintained reasonably constant even in the case of insufficient solar radiation, by virtue of the flexibility of operation afforded by the system of automatic valves described above, which enables path of flows of different length to be obtained, as described previously.

The solution to be gasified is loaded from one end of the reactor and passes right through it (i.e., along the entire network of converters 1, 100, 200, 300, 400) inside the high-pressure flow conduits 42, 142, 242, 342, 442. Here the solution is heated thanks to the incident solar radiation (ISR) concentrated by the mirrors PM1-PM4 until it exceeds the critical temperature of the water and reaches 550-600° C., a temperature at which it is possible to deem that the gasification reactions are triggered.

By keeping the mixture in these conditions for a pre-set time, around 1 to 2 minutes, and continuing to supply energy via solar radiation, it is possible to obtain conversions of the organic matter up to 80%, with high yields in methane and hydrogen.

From the instant when a certain minimum threshold temperature is reached, which in general depends upon the nature and composition of the waste fed, the gasification reactions start to take place within the gasification reactor 500, namely, thermal decomposition of the organic molecules, which react with the water that constitutes the reaction environment to form simple molecules such as methane, hydrogen, carbon monoxide and carbon dioxide, hydrogen sulphide (if the incoming waste contains sulphur), and inert salts, according to the generic gasification reaction referred to previously.

Located at output from the reactor 500, i.e., at the opposite end with respect to the supply end, is a trap for ash 607, which has the function of accumulating and discharging, in an automated way at regular time intervals, the carbon residue, the ashes, including and the salts, which have formed within the reactor 500.

The line downstream of the trap 607 traverses a first cooling stage consisting in a device for accumulating thermal energy (thermal energy accumulator device) 609, preferably of the type described in the patent application for industrial invention No. 102017000091905, filed on Aug. 8, 2017), transferring thereto an amount of thermal energy that is accumulated and released for heating a service fluid (for example, a diathermic oil) for use in an ORC system, operatively connected to an alternator 610 for the production of electrical energy. After passing through the thermal energy accumulator device 609, the line downstream of the trap 607 gives out into a static trap for H₂S, designated by the reference 611, having the purpose of capturing and converting the possible hydrogen sulphide produced during gasification (in the case where the waste supplied contains sulphur).

The trap 611 comprises a thermally insulated tube filled with a bed of adsorbent material, for example, with a base of iron oxides (or alternatively also zinc oxides), capable of converting the hydrogen sulphide H₂S into ferrous sulphide and releasing water according to the following reaction:

H₂S+FeO→FeS+H₂O

A second reaction unit 613, which is also adiabatic and of a tubular type, filled with pellets or powdered alumina or else chromium or magnesium oxides, impregnated with a specific transition metal (e.g., nickel, cobalt, iron, ruthenium, rhodium, molybdenum, etc.) performs the function of catalytic methanator; i.e., it converts the hydrogen and carbon oxides into methane, according to the following set of reactions:

CO₂+H₂→CO+H₂O reaction of displacement of water vapour:

CO+3H₂→CH₄+H₂O methanation reaction:

Given that the methanation process is globally exothermic and conducted in (ideally) adiabatic conditions, the current will undergo an increase in temperature. The thermal energy available in the effluent is used for pre-heating the current entering the methanator itself, in the perspective of energy optimisation of the plant, via a heat exchanger operating at high pressure and high temperature, designated by the reference 612. The latter is preferably obtained as described in the patent application for invention No. 102016000009566, filed on Jan. 29, 2016 and in the international patent application No. PCT/IB2017/050445, filed on Jan. 27, 2017.

The high-pressure section of the plant terminates with a gas-liquid separator 615, where the relatively cold biogas current arrives slightly laminated (thermal-expansion valve 614), thus cooling further by the Joule-Thomson effect.

The pressure, which is still sustained, and the temperature, which is by now low (with respect to the process conditions) enables a fair share of the carbon dioxide present to be withheld in the aqueous phase.

Here, two phases are separated, namely:
the condensed aqueous share, which is further laminated (thermal-expansion valve 616) down to a pressure just a little higher than atmospheric pressure and sent to a second atmospheric phase separator 617;
the gaseous share, which in this step constitutes the raw biogas proper (methane˜70%, carbon dioxide˜30%); this is passed through medium-pressure phase separator 618 (operating at 15-20 bar), prior to being sent on for biogas upgrading by means of a membrane separation system 619.

The biogas-upgrade system based upon membrane technology is commercially available and is usually constituted by a series of filters 620 for removal of humidity, possible traces of oil, and fine particulate, an electric heater 621, and one or more membrane separation modules 622, possibly with a dual-stage arrangement with recirculation to increase recovery of methane. The membrane separator 622, according to its configuration, can produce a retentate current enriched in methane with levels of purity even higher than 98% and a permeate current, which is chiefly constituted by carbon dioxide but may contain even more than 20% of methane, according to the plant configuration. The retentate current represents the useful product, which can be sent into the grid; the permeate current, passing through the atmospheric phase separator 617, is sent into a boiler 623.

Combustion in the boiler 623 of the resulting current, prevalently constituted by the permeate of the membrane system (in which the sole fuel present is methane), enables exhaust EXH into the atmosphere, as in the case of ordinary civil forms of exhaust. Prior to discharge into the atmosphere, the gas is in any case scrubbed in a scrubbing column 624, where it is in contact in countercurrent with the alkaline solution GS coming from the basic-hydrolysis tank 605, which abates any traces that may be present of other acidic gases, hence carrying out scrubbing of the exhaust gas, which will basically be made up of carbon dioxide alone.

The boiler 623 at the same time heats a closed-circuit current of diathermic oil 625, which in turn transfers heat to a further, final, thermal energy accumulator device 626 (once again obtained preferentially like the thermal energy accumulator device 609) for the production of low-pressure steam VAP necessary for supplying the serpentine installed in the thermal-hydrolysis tank 602.

Finally, the aqueous effluent of the atmospheric phase separator 617, net of the aliquots drawn off and recirculated into the thermal-hydrolysis tank 602 and into the acid-hydrolysis tank 604, is sent on to a second neutralisation tank NT2, where corresponding neutralisation reagents (flow RNT2) are added, before it can be discharged into the sewage system (flow WP).

Generalising, the plant 600 may be broken down into the following scheme, which includes in particular:
a pre-treatment section (which includes the assembly 601, the tanks 602, 604, 605, and the centrifugal separator 603) configured to obtain, starting from the organic fraction of municipal solid waste (OFMSW), an organic aqueous solution (with low content of suspended solids);
the solar reactor 500;
a feeder (pump 606) for feeding the organic aqueous solution to the solar reactor 500; the reactor 500 is configured for carrying out a thermal treatment of the aforesaid organic aqueous solution, including gasification in supercritical water, cracking, and reforming (these latter processes take place in the vapour phase); and
an after-treatment section for treatment of the products of reaction of the solar reactor 500 (including the entire set of devices 607-626 downstream of the reactor 500) configured for extracting methane from said products of reaction.

Moreover, the post-treatment section includes one or more devices for accumulating thermal energy (thermal energy accumulator devices 609, 626) configured for extracting thermal energy from the products of reaction of the reactor 500.

One of the above devices (and if so required, even more than one) for accumulating thermal energy set downstream of the solar reactor 500 is moreover configured for operating as heater for a service fluid of an ORC system by release of the thermal energy accumulated by extraction from the products of reaction. In the plant 600 this is, in particular, the thermal energy accumulator device 609.

The converter 1, 100, 200, 300, 400 hence operates as solar-energy chemical reactor capable of carrying out conversion of the organic fraction of municipal solid waste into biofuel (methane) and recovering the excess heat using accumulators (thermal energy accumulator devices).

The heat accumulated is then converted into electrical energy, with the triple final result of:
disposal of the OFMSW;
production of electrical energy;
production of methane with high yields (an anaerobic digestor has yields not higher than 8%) that are to be sent into the power grid for civil and industrial purposes; and
production of water for irrigation purposes (the so-called clean water).

The advantages referred to above become even more evident, reducing the problems of cost/competitiveness, if it is considered that the converter 1, 100, 200, 300, 400 can be obtained, as has been seen, by simple modification of an ordinary receiver tube for thermodynamic solar plants.

Of course, the details of construction and the embodiments may vary widely with respect what has been described and illustrated herein, without thereby departing from the sphere of protection of the present invention, as defined by the annexed claims.

1. A solar energy converter device (1; 100; 200; 300; 400), comprising:
a shell (2; 102; 202; 302; 402), and
a core (4; 104; 204; 304; 404) internal to said shell (2; 102; 202; 302; 402),
wherein said shell (2; 102; 202; 302; 402) and said core (4; 104; 204; 304; 404) develop axially along a longitudinal axis (X) and comprise a volume therebetween,
said core (4; 104; 204; 304; 404) including a thermally conductive matrix (41; 141; 241; 341; 441) in thermal exchange relationship with said volume, and
said matrix (41; 141; 241; 341; 441) housing one or more flow conduits (42; 142; 242; 342; 442) for a working fluid, said one or more flow conduits (42; 142; 242; 342; 442) being in a thermal exchange relationship with said thermally conductive matrix (41; 141; 241; 341; 441).

2. The solar energy converter device (1; 100; 200; 300; 400) according to claim 1, wherein vacuum is provided inside said volume.

3. The solar energy converter device (1; 100; 200; 300; according to claim 1, wherein said shell (2; 102; 202; 302; 402) is made of refractive material.

4. The solar energy converter device (1; 100; 200; 300; 400) according to claim 3, wherein said shell (2; 102; 202; 302; 402) comprises a coating made of a solar radiation adsorbent material.

5. The solar energy converter device (1; 100; 200; 300; 400) according to claim 1, wherein:
said shell (2; 102; 202; 302; 402) is a tubular shell,
said matrix (41; 141; 241; 341; 441) extends along said longitudinal axis (X) from a first end to a second end of said tubular shell (2; 102; 202; 302; 402), and
said one or more flow conduits (42; 142; 242; 342; 442) extend within said matrix (41; 141; 241; 341; 441) along said longitudinal axis (X) and give out in correspondence of said first and second ends, and
a first end member (6; 106; 206; 306; 406) and a second end member (8; 108; 208; 308; 408) are provided respectively at said first end and said second end and delimit the volume between the tubular shell (2; 102; 202; 302; 402) and the core (4; 104; 204; 304; 404).

6. The solar energy converter device (1; 100; 200; 300; according to claim 1, wherein:
said shell (2; 102; 202; 302; 402) comprises an outer tubular element, and
said core (4; 104; 204; 304; 404) comprises an inner tubular element wherein said matrix (41; 141; 241; 341; 441) is housed.

7. The solar energy converter device (1; 100; 200; 300; 400) according to claim 5, wherein said matrix (441) is made of a compacted powdered material.

8. The solar energy converter device (1; 100; 200; 300; according to claim 1, wherein said one or more flow conduits (42; 142; 242; 342, 442) are resistant to internal pressures higher than 221 bars, are resistant to continued operation temperatures higher than 374° C., and are resistant to aggressive and embrittling chemical agents.

9. A solar reactor (500)₁ comprising:
a solar energy converter device (1; 100; 200; 300; 400) according to claim 1, and
a solar radiation concentrator device including one or more reflecting elements configured to concentrate the incident solar radiation in correspondence of said solar energy converter device (1; 100; 200; 300; 400).

10. The solar reactor (500) according to claim 9, wherein said concentrator device includes at least one of:
a Fresnel mirror array (PM; PM1, PM2, PM3, PM4),
a linear parabolic collector, and
a disc parabolic collector.

11. The solar reactor (500) according to claim 9, including a plurality of said converter devices (1, 100, 200, 300, 400) hydraulically connected to each other, the hydraulic connection including a parallel connection of converter devices (1, 100, 200, 300, 400) which are in turn hydraulically connected in series, wherein in one or more series of converter devices (1, 100, 200, 300, 400), one or more flow direction and control valves are installed which are configured to establish a hydraulic connection branching off the corresponding series with one or more further series.

12. The solar reactor (500) according to claim 11, wherein said one or more flow direction and control valves are electrically or pneumatically controllable and can be monitored by means of a computer.

13. A plant (600) for the treatment of an organic fraction of urban solid wastes (FORSU), comprising:
a pre-treatment section configured for converting said organic fraction of urban solid wastes (FORSU) into an aqueous organic solution;
a solar reactor (500) according to any of claims 9 to 12,
a feeder (606) of said aqueous organic solution to said solar reactor (500), said solar reactor (500) being configured for thermal treatment of the aqueous organic solution, and
an after-treatment section for reaction products of said solar reactor (500) configured for extracting methane from said reaction products.

14. The plant (600) according to claim 13, wherein said after-treatment section includes one or more thermal energy accumulator devices configured for harvesting thermal energy from said reaction products.

15. The plant (600) according to claim 14, wherein a thermal energy accumulator device (609) arranged downstream of said solar reactor (500) is furthermore configured to operate as a heater for a service fluid of an organic Rankine Cycle plant (ORC) by releasing thermal energy stored through harvesting from the reaction products.
